(19) United States
(12) Patent Application Publication
(10) Pub. No.: US 2013/0171640 A1
(43) Pub. Date: Jul. 4, 2013

(54) SOLID REAGENT DISSOLVING DEVICE AND METHOD OF DISSOLVING SOLID REAGENT BY USING THE SAME

(71) Applicant: Samsung Electronics Co., Ltd., Suwon-si (KR)

(72) Inventors: Sung-hong KWON, Yongin-si (KR); Sung-ouk JUNG, Hwaseong-si (KR); Sung-min CHI, Hwaseong-si (KR); Kyu-youn HWANG, Seoul (KR); Joon-ho KIM, Seongnam-si (KR)

(73) Assignee: SAMSUNG ELECTRONICS CO., LTD., Suwon-si (KR)

(21) Appl. No.: 13/721,947

(22) Filed: Dec. 20, 2012

(30) Foreign Application Priority Data
Dec. 29, 2011 (KR) 10-2011-0146104

(51) Int. Cl. G01N 1/38; B01L 3/00; C12Q 1/68

(52) U.S. Cl. CPC G01N 1/38; C12Q 1/6806; B01L 3/5027
USPC 435/6.11; 435/6.12; 436/63; 436/86; 436/174; 422/503

(57) ABSTRACT

A solid reagent dissolving device including a flexible layer; an upper plate disposed on the flexible layer; and a lower plate disposed under the flexible layer, wherein the upper plate comprises a plurality of minute channels, a dissolution chamber connected with the plurality of minute channels, and a protrusion for limiting a flow of a fluid flowing through one of the plurality of minute channels, the lower plate comprises a plurality of penetration holes that correspond to the protrusion and the dissolution chamber, respectively, and one side of each of the plurality of penetration holes, the plurality of minute channels, and the dissolution chamber are covered with the flexible layer, and method of using same.

### CROSS-REFERENCE TO RELATED APPLICATIONS

This application claims the benefit of Korean Patent Application No. 10-2011-0146104, filed on Dec. 29, 2011, in the Korean Intellectual Property Office, the disclosure of which is incorporated herein in its entirety by reference.

### BACKGROUND

1. Field

The present disclosure relates to micro-devices that are used in molecular diagnostic equipment, and more particularly, to a solid reagent dissolving device and a method of dissolving a solid reagent by using the solid reagent dissolving device.

2. Description of the Related Art

Diagnostic equipment has been more and more miniaturized and automated due to the demands for safety and user convenience and fast point of care testing (POCT).

A liquid reagent is difficult to keep, and the stability thereof is relatively low. On the other hand, the stability of a solid reagent or a lyophilized reagent is relatively high, and thus, the solid reagent or the lyophilized reagent has a relatively long shelf life. In addition, the volume of the solid reagent or the lyophilized reagent may be reduced, and thus, the size of a storage container for keeping the solid reagent or the lyophilized reagent is relatively small. Thus, in miniaturized and automated diagnostic equipment, the solid reagent or the lyophilized reagent is mainly used.

In the diagnostic equipment, the solid reagent or the lyophilized reagent has to be dissolved into liquid to react with any other reagent and detect a signal.

Many studies of methods of mixing different kinds of solutions in a micro-device have been performed. However, few studies of methods of dissolving a solid reagent in a micro-device exist.

### SUMMARY

Provided are solid reagent dissolving devices that are capable of reducing dissolution time of a solid reagent and improving reproducibility thereof.

Provided are methods of dissolving a solid reagent by using the solid reagent dissolving device.

Additional aspects will be set forth in part in the description which follows and, in part, will be apparent from the description, or may be learned by practice of the presented embodiments.

According to an aspect of the present invention, a solid reagent dissolving device includes: a flexible layer; an upper plate disposed on the flexible layer; and a lower plate disposed under the flexible layer, wherein the upper plate includes a plurality of minute channels, a dissolution chamber connected with the plurality of minute channels, and a protrusion for limiting a flow of a fluid flowing through one of the plurality of minute channels, the lower plate includes a plurality of penetration holes that correspond to the protrusion and the dissolution chamber, respectively, and one side of each of the plurality of penetration holes, the plurality of minute channels, and the dissolution chamber are covered with the flexible layer.

A portion corresponding to the dissolution chamber in the upper plate may include a cover in which the solid reagent is placed.

A portion corresponding to the dissolution chamber in the upper plate may be parallel with the flexible layer.

Diameters of both sides of each of the plurality of penetration holes may be equal to or different from each other.

A penetration hole corresponding to the protrusion may include a valve chamber for opening and closing a path between the protrusion and the flexible layer.

At least one of the penetration holes may correspond to the dissolution chamber, and the at least one of the penetration holes may include a pneumatic chamber that generates a vibration of a portion, which corresponds to the dissolution chamber, in the flexible layer.

Physical properties of a surface of the flexible layer, surfaces of the plurality of minute channels, and an internal side of the dissolution chamber, with respect to the fluid that is input through one of the plurality of minute channels, may be the same as or different from each other.

The cover may be separable from the upper plate, and the internal side of the cover may include at least one curved surface portion in which a solid reagent is placed.

The cover may include first and second covers that are apart from each other, and internal sides of the first and second covers may include respective curved surface portions in which different solid reagents are placed.

The respective curved surface portions may be convex upward or downward.

According to another aspect of the present invention, a method of dissolving a solid reagent includes: disposing the solid reagent in a dissolution chamber; supplying a solution for dissolving the solid reagent to the dissolution chamber; and vibrating the solution for dissolving.

The solid reagent may be a reagent solidified by drying a liquid reagent. The solid reagent may be a lyophilized reagent.

The disposing of the solid reagent may include locating a previously prepared solid reagent in a location where the solid reagent is disposed in the dissolution chamber. The locating of the solid reagent may be performed by injecting the solid reagent through a minute channel connected to the dissolution chamber. Otherwise, the locating of the solid reagent may be performed by separating a portion of the dissolution chamber, introducing the solid reagent into the separated portion, and then combining again the separated portion, into which the solid reagent has been introduced, with the remaining portion of the dissolution chamber. Thus, a portion of the dissolution chamber may be separable. In addition, the separable portion of the dissolution chamber and the remaining portion of the dissolution chamber may be combined by using a combining means, for example, a mechanical combining means or an adhesive.

The disposing of the solid reagent may include: disposing a liquid reagent at a location where the solid reagent is disposed in the dissolution chamber; and lyophilizing the liquid reagent.

The disposing of the liquid reagent may include introducing the liquid reagent into the dissolution chamber. The introducing of the liquid includes introducing the liquid reagent through the minute channel connected to the dissolution chamber. In addition, the introducing of the liquid may be performed by separating a portion of the dissolution chamber, introducing the liquid reagent into the separated portion, and then combining again the separated portion, into which the liquid reagent has been introduced, with the remaining portion of the dissolution chamber.

The lyophilizing of the liquid reagent may be performed in the state in which the liquid reagent has been introduced into the dissolution chamber or may be performed by separating a portion of the dissolution chamber, introducing the liquid reagent into the separated portion, and lyophilizing the liquid reagent introduced into the separated portion. The reagent lyophilized in the separated portion may be finally located in the dissolution chamber by combining again the separated portion with the remaining portion of the dissolution chamber. The lyophilizing may be performed by using a known method or apparatus.

As stated above, the method of dissolving a solid reagent includes supplying a solution for dissolving the solid reagent to the dissolution chamber. The solution for dissolving may have a characteristic for dissolving the solid reagent. The solution for dissolving may include water, a saline solution, and/or a buffer. The buffer may be properly selected depending on a selected reagent. The buffer may be a phosphate buffer solution (PBS) or a tris(hydroxymethyl)aminomethane (Tris) buffer. The supplying of the solution may include letting the solution flow through a minute channel connected to the dissolution chamber.

The vibrating of the solution for dissolving may include vibrating a flexible layer covering the dissolution chamber.

The flexible layer may be vibrated with a frequency in the range of about 0.001 Hz to about 100 k Hz.

The vibrating of the flexible layer may include repeating a process of raising or lowering a pressure under the flexible layer compared to when the flexible layer does not vibrate.

The vibrating of the solution for dissolving may include vibrating the solid reagent as well as the solution for dissolving.

The method of dissolving a solid reagent may further includes, before the vibrating of the solution, blocking at least one portion of a minute channel connected to the dissolution chamber.

The blocking of the at least one portion of the minute channel may include pressuring a portion of a flexible layer covering the minute channel that is blocked.

The solution may include a target material that reacts with the solid reagent, and the target material may be a target DNA. For example, the solid reagent may be a lyophilized PCR reagent, and the solution may dissolve a lyophilized polymerase chain reaction (PCR) reagent and may include a template DNA that may react with the PCR reagent. The target material may include a target RNA, a protein, or a cell debris. The PCR reagent may include polymerase, a primer/probe, a dNTP, and a buffer. The solid reagent may be a lyophilized nucleic acid hybridization reagent, a ligation reaction reagent, a restriction enzyme reaction reagent, an in vitro transcription reaction reagent, or an in vitro translation reaction reagent.

The dissolution chamber may include beads that vibrate with the solution and are used for dissolving the solid reagent. The beads may be microbeads that are capable of being included in the dissolution chamber 48. The microbeads may have a diameter in the range of about 10 nm to about 1000 um.

A portion of the dissolution chamber may be a cover, the cover may be separable from the dissolution chamber, and an internal side of the cover may include at least one curved surface portion in which a liquid reagent is placed.

At least one pneumatic chamber that is used for vibrating the solution for dissolving may correspond to the dissolution chamber.

The cover may include first and second covers that are apart from each other, and internal sides of the first and second covers may include respective curved surface portions in which different liquid reagents are placed.

In the solid reagent dissolving device, a solid reagent is dissolved by vibrating a flexible intermediate layer located in a boundary between a dissolution chamber and a pneumatic chamber. By dissolving the solid reagent by using such a dynamic method, dissolution time of the solid reagent may be reduced, and the solid reagent may be more completely dissolved, thereby improving reproducibility thereof. In addition, the dissolution time may be further reduced by using beads in a dissolving process, and the reproducibility may be further improved. Thus, by applying the solid reagent dissolving device to various molecular diagnostic equipment, in which a process of dissolving the solid reagent or a lyophilized reagent is necessary, for example, polymerase chain reaction (PCR) equipment or external diagnostic equipment, diagnosis time may be reduced, and reliability of diagnosis may be improved.

### BRIEF DESCRIPTION OF THE DRAWINGS

These and/or other aspects will become apparent and more readily appreciated from the following description of the embodiments, taken in conjunction with the accompanying drawings in which:

FIG. 1 is a cross-sectional view of a solid reagent dissolving device according to an embodiment of the present invention;

FIG. 2 is a plan view of a bottom side of an upper plate of the device of FIG. 1;

FIG. 3 is a side view taken along the line 3-3′ of FIG. 2;

FIG. 4 is a cross-sectional view illustrating a case where a plurality of chambers are formed under a dissolution chamber of FIG. 1;

FIG. 5 is a cross-sectional view of a solid reagent dissolving device according to an embodiment of the present invention;

FIG. 6 is a cross-sectional view illustrating a case where a second cover is disposed instead of a first cover of FIG. 5;

FIG. 7 is a plan view illustrating a case where two covers are disposed in an upper plate of a dissolution chamber in a solid reagent dissolving device according to an embodiment of the present invention;

FIG. 8 is a cross-sectional view taken along the line 8-8′ of FIG. 7;

FIG. 9 is a cross-sectional view illustrating a case where a plurality of pneumatic chambers are formed instead of a second chamber of FIG. 8;

FIG. 10 is a cross-sectional view illustrating a case where third and fourth covers of FIG. 8 are replaced with different types of covers;

FIG. 11 is a cross-sectional view illustrating a case where third and fourth covers of FIG. 9 are replaced with different types of covers;

FIG. 12 is a cross-sectional view of a solid reagent dissolving device according to an embodiment of the present invention;

FIG. 13 is a cross-sectional view illustrating a case where a plurality of pneumatic chambers are formed in the solid reagent dissolving device of FIG. 12;

FIGS. 14 through 18 are cross-sectional views illustrating, in stages, a method of dissolving a solid reagent, according to an embodiment of the present invention; and

FIGS. 19 through 21 are cross-sectional views illustrating, in stages, a method of dissolving a solid reagent, according to an embodiment of the present invention.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

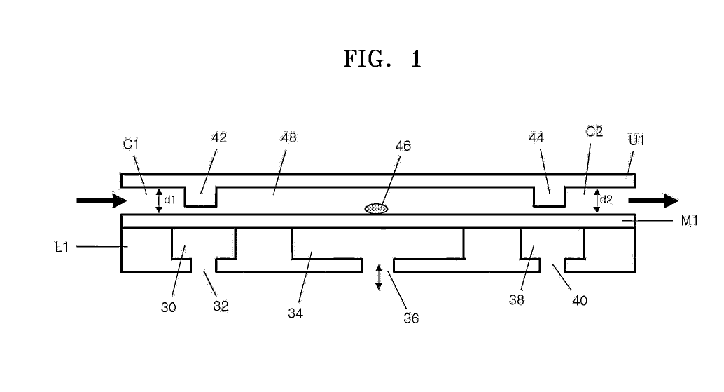

FIG. 1 is a cross-sectional view of a solid reagent dissolving device (“dissolving device”) according to an embodiment of the present invention.

Referring to FIG. 1, the dissolving device having a three-layer structure includes a lower plate L1, an upper plate U1, and a flexible intermediate layer M1 disposed between the lower plate L1 and the upper plate U1. The material of the lower plate L1 may be silicon, glass, plastic, or any other suitable material. The lower plate L1 includes a plurality of chambers, for example, first through third chambers 30, 34, and 38. The first through third chambers 30, 34, and 38 may be penetration holes of which upper and lower sides are open, and upper openings in the first through third chambers 30, 34, and 38 are covered with the flexible intermediate layer M1. Lower openings 32, 36, and 40 of the first through third chambers 30, 34, and 38 are inlets and outlets of pressure, e.g., air pressure. In the first through third chambers 30, 34, and 38, diameters of the upper openings may be greater than, less than, or equal to those of the lower openings 32, 36, and 40. An internal space of the second chamber 34 may be greater than or less than those of the first and third chambers 30 and 38. In addition, the internal spaces of the first through third chambers 30, 34, and 38 may be equal to each other. The internal spaces of the first and third chambers 30 and 38 may be equal to or different from each other.

Pressure—such as air pressure—may be applied to the first chamber 30 causing the flexible intermediate layer M1 to contact a first protrusion 42 of the upper plate U1 and close a channel formed between the intermediate layer M1 and the first protrusion 42. Similarly, applying pressure to the third chamber 38 may cause the intermediate layer M1 to contact a second protrusion 44 of the upper plate U1, and close a channel formed between the intermediate layer M1 and the second protrusion 44. If the pressure applied to the first and third chambers 30 and 38 is removed or reduced, the closed channel between the intermediate layer M1 and the first protrusion 42 and the closed channel between the intermediate layer M1 and the third protrusion 44 may be opened. In this manner, since the channel between the intermediate layer M1 and the first protrusion 42 and the channel between the intermediate layer M1 and the second protrusion 44 are closed or opened, the first and third chambers 30 and 38 may be pressure valve chambers.

The second chamber 34 may be a pneumatic chamber in which pressurization (e.g., pressure higher than atmosphere pressure) and depressurization (e.g., pressure lower than atmosphere pressure) using a fluid—such as air—are periodically and repeatedly performed. If pressure is applied to the second chamber 34 through the lower opening 36 of the second chamber 34, which is an inlet, the intermediate layer M1 may become convex upwards. On the contrary, if the second chamber 34 is depressurized, the intermediate layer M1 may become concave. Thus, periodic and repeated pressurization and depressurization of the second chamber 34 may cause the intermediate layer M1 to vibrate up and down. In some embodiments, the intermediate layer M1 and/or a contact side of the intermediate layer M1—which contacts a fluid—has one or more physical properties that facilitate smooth fluid flow according to the type of fluid. For example, the contact side of the intermediate layer M1 may be hydrophilic, hydrophobic, or have other physical properties that facilitate smooth fluid flow. The intermediate layer M1 may be a polymer layer, and a thickness thereof may be from about 1 μm to about 1000 μm, for example, about 1 μm˜500 μm. The polymer layer may be, for example, a polydimethylsiloxane (PDMS) layer, a poly(methyl methacrylate) (PMMA) layer, a polypropylene (PP) layer, a polycarbonate (PC) layer, a cyclic olefin copolymer (COC) layer or a polyurethane (PU) layer. A solid reagent 46 may be located on the intermediate layer M1 over the second chamber 34. The solid reagent 46 may be located over the lower opening 36 of the second chamber 34, which is an inlet. The solid reagent 46 may be a reagent solidified by drying a liquid reagent. For example, the solid reagent 46 may be a lyophilized reagent.

An external side (upper side) of the upper plate U1 may be a flat plane and may be parallel with the intermediate layer M1. The upper plate U1 includes first and second minute channels C1 and C2, the first and second protrusions 42 and 44, and a dissolution chamber 48. A portion of the upper plate U1, which defines the dissolution chamber 48, is parallel with the intermediate layer M1. The first and second protrusions 42 and 44 are spaced apart from each other. The dissolution chamber 48 is located between the first and second protrusions 42 and 44. The first protrusion 42 is located around the first minute channel C1. The second protrusion 44 is located around the second minute channel C2. The first and second protrusions 42 and 44 protrude toward the intermediate layer M1. The first protrusion 42 is located over the first chamber 30 of the lower plate L1. The second protrusion 44 is located over the third chamber 38 of the lower plate L1.

Lengths of the first and second protrusions 42 and 44 are equal to or different from each other. The length of the first protrusion 42 is shorter than a depth d1 of the first minute channel C1. A depth d2 of the second minute channel C2 may be equal to the depth d1 of the first minute channel C1. The depths d1 and d2 of the first and second minute channels C1 and C2 may be different from each other. In this manner, there is a gap between the first protrusion 42 and the intermediate layer M1 due to a difference between the depth d1 of the first minute channel C1 and the length of the first protrusion 42, and there is a gap between the second protrusion 44 and the intermediate layer M1 due to a difference between the depth d2 of the second minute channel C2 and the length of the second protrusion 44.

The solid reagent dissolving device and components thereof, including the penetration holes forming valve chambers, dissolution chamber, and minute channels, may have any suitable volumes or dimensions. In some embodiments, the penetration holes may have a length equal to the thickness of the lower plate (e.g., about 1 μm˜10 cm) and a maximum diameter of about 1 μm˜10 cm; the minute channels may have a maximum diameter of about 1 μm˜1 cm; the dissolution chamber may have a volume of about 1 nl˜10 ml (e.g., about 1 ul˜100 ul); and the upper plate may have a dimension at its maximum thickness of about 1 μm˜10 cm.

In this embodiment, applying a certain amount of pressure to the first and third chambers 30 and 38 causes the intermediate layer M1 to contact protrusions 42 and 44. Consequently, fluid that is input through the first minute channel C1 cannot flow into the dissolution chamber 48, and fluid in the dissolution chamber 48 cannot be discharged into the second channel C2. Similar to the intermediate layer M1, a contact side of the upper plate U1, and/or surfaces of the first and second minute channels C1 and C2, and/or an internal side of the dissolution chamber 48 may have one or more physical properties that facilitate smooth fluid flow. Physical properties of the surfaces of the first and second minute channels C1 and C2, the surface of the intermediate layer M1, and the internal side of the dissolution chamber 48 with respect to the fluid may be the same as or different from each other. Accordingly, generation of bubbles may be minimized when a fluid flows into the dissolution chamber 48.

In some embodiments, the fluid introduced into the dissolving device may be a solution for dissolving a solid reagent. For example, the solution may dissolve a lyophilized polymerase chain reaction (PCR) reagent, and may include a template DNA that may react with the PCR reagent. The solid reagent 46 may be located or disposed on the intermediate layer M1 inside the dissolution chamber 48.

In the example embodiment of FIG. 1, the left arrow (proximate the first minute channel C1) indicates a fluid that is input through the first minute channel C1, and the right arrow (proximate the second minute channel C2) indicates a fluid that is discharged from the dissolution chamber 48 through the second minute channel C2.

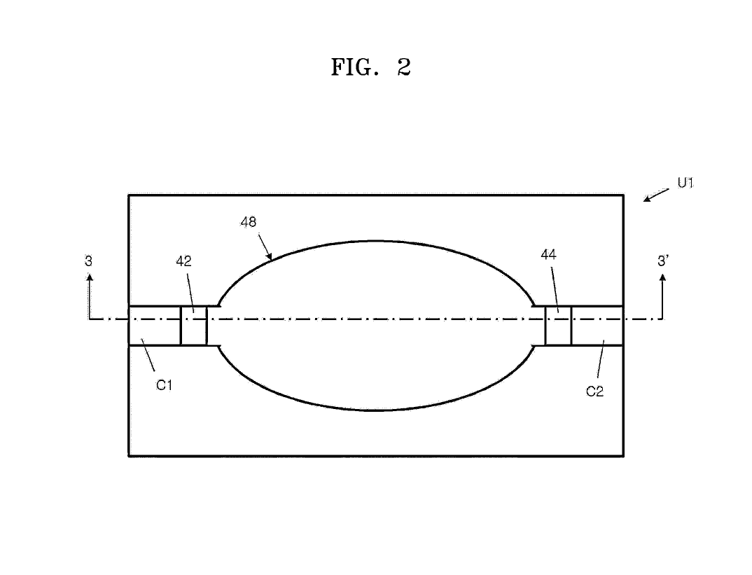

FIG. 2 is a plan view of the bottom side of the upper plate U1. In the example embodiment of FIG. 2, the dissolution chamber 48 includes a plane of an elliptical shape, however the shape of the plane is not limited thereto. The plane of the dissolution chamber 48 may have a round shape, a tetragonal shape, or other polygonal shapes. As illustrated, the first and second protrusions 42 and 44 are adjacent to the dissolution chamber 48.

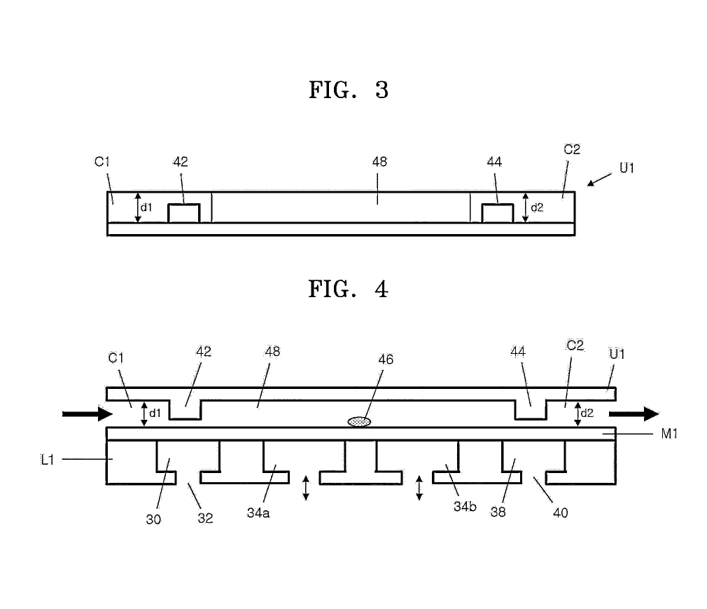

FIG. 3 is a side view taken along the line 3-3′ of FIG. 2. Referring to FIG. 3, the lengths (or heights) of the first and second protrusions 42 and 44 may be shorter than the depths d1 and d2 of the first and second minute channels C1 and C2.

In the example embodiment of FIG. 4, the second chamber 34 of FIG. 1 is divided into, is replaced by, or comprises a plurality of chambers. In this example the second chamber 34 is divided into fourth and fifth chambers 34a and 34b. The fourth and fifth chambers 34a and 34b are apart from each other and located under the dissolution chamber 48. The fourth and fifth chambers 34a and 34b may be connected to separate, respective pumps (e.g., air pumps), or may be commonly connected to a single pump. While FIG. 4 illustrates the second chamber 34 (of FIG. 1) as divided into two chambers, the present disclosure is not limited thereto. Thus, the second chamber 34 of FIG. 1 may be divided into more than two chambers, e.g., three, four, five, six, seven, and so on. Each chamber may be connected to a separate pump, or the chambers may be connected to a common pump.

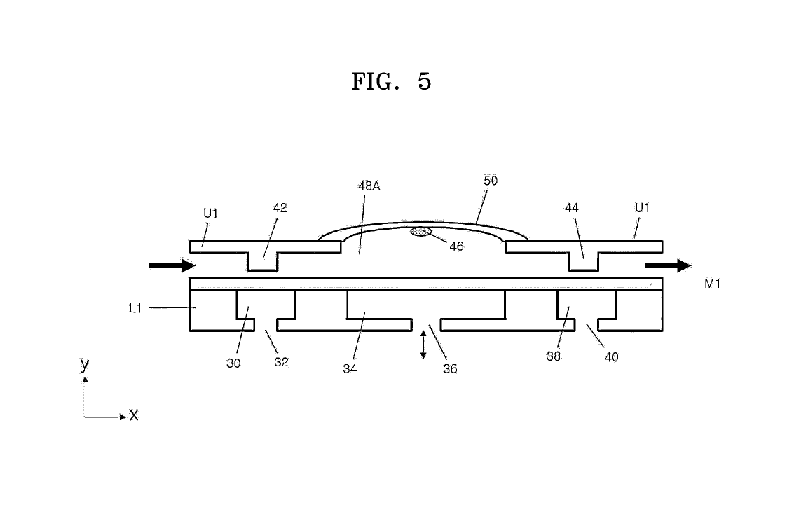

FIG. 5 illustrates a cross-sectional view of another example embodiment of a dissolving device. A description of features similar to those described in FIG. 1 is not repeated; only features different from the dissolving device of FIG. 1 are described.

In the example dissolving device of FIG. 5, a portion of an upper plate U1 over the second chamber 34 is removed and covered with a first cover 50. In other words, the second chamber is exposed, in part, through the upper plate and the exposed portion covered by a first cover that, when present, defines part of the dissolution chamber. Due to the first cover 50, an external side (upper side) of the upper plate U1 includes a curved surface portion that is not parallel with an intermediate layer M1. In addition, due to the first cover 50, a dissolution chamber 48A includes a portion that is not parallel with the intermediate layer M1.

The dissolving device of FIG. 1 has a three-layer structure, whereas the dissolving device of FIG. 5 has a four-layer structure by further including the first cover 50. The shape of the first cover 50 may be a semicircular, elliptical, tetragonal, polygonal, or any other desired shape. In some embodiments, the first cover 50 is curved such that a central portion of the first cover 50 extends away from the dissolution chamber 48A, which may increase the volume of the dissolution chamber 48A of FIG. 5 as compared to the volume of the dissolution chamber 48 of FIG. 1. In some embodiments, the external side of the first cover 50 may be considered convex in the Y-axis direction, and the internal side of the first cover 50—which contacts a fluid or solution that flows into the dissolution chamber 48A—may be considered concave in the Y-axis direction.

As illustrated in FIG. 5, when the first cover 50 is disposed on the dissolving device, the internal side or at least a portion of the internal side of the first cover 50 may be higher than the upper (exterior) side of the upper plate U1. In the dissolving device of FIG. 5, a solid reagent 46 may be located underneath the internal side of the cover 50. The solid reagent 46 may be located at the top of the internal side of the cover 50. While the first cover 50 is depicted as curving away from and increasing the volume of the dissolution chamber 48A, the first cover 50 may curved toward and decreasing the volume of the dissolution chamber 48A.

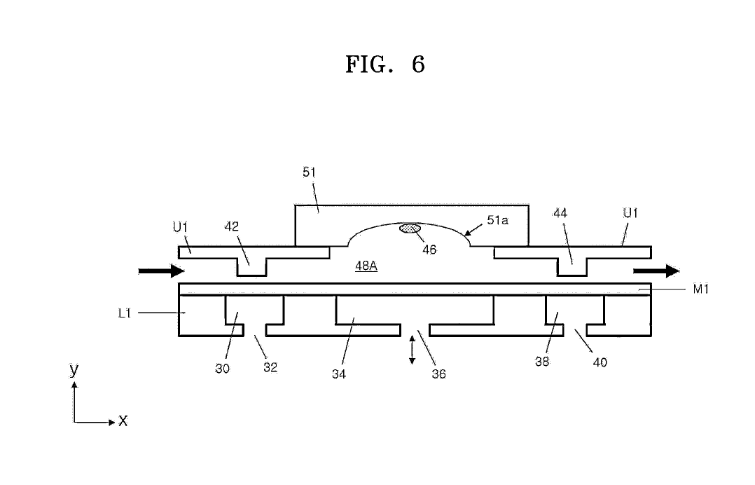

The example embodiment of FIG. 6 illustrates a second cover 51, in place of the first cover 50, disposed on the dissolving device. In this embodiment, the upper side of the second cover 51 is parallel with the upper side of the upper plate U1; the lateral sides of the second cover 51 are perpendicular to the upper side of the upper plate U1; and the internal side of the second cover 51 that contacts a fluid or solution that flows into the dissolution chamber 48A includes a curved surface portion 51a. The curved surface portion 51a may be concave in the Y-axis direction. The solid reagent 46 may be located at the top of the curved surface portion 51a.

In FIG. 6, the third and fourth chambers 34a and 34b illustrated in FIG. 4 may be formed instead of the second chamber 34.

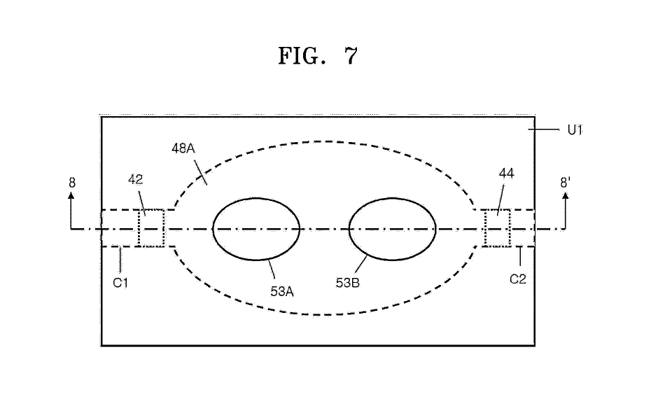

The upper plate U1 may include a plurality of curved surface portions. FIG. 7 illustrates a case where two covers, that is, third and fourth covers 53A and 53B, are disposed on the upper plate U1. While FIG. 7 illustrates the upper plate U1 as including two curved surface portions, the present disclosure is not limited thereto. Thus, the upper plate U1 may be divided into more than two curved surface portions, e.g., three, four, five, six, seven, and so on.

Referring to FIG. 7, the third and fourth covers 53A and 53B are spaced apart from each other. The third and fourth covers 53A and 53B may be aligned in the X-axis direction, the Y-axis direction, or another direction (axial directions are depicted in FIG. 6). The size, shape, and volume of the third and fourth covers 53A and 53B may be equal or different. The plane shapes of the third and fourth covers 53A and 53B may be round, tetragonal, polygonal, elliptical, or any other desired shape.

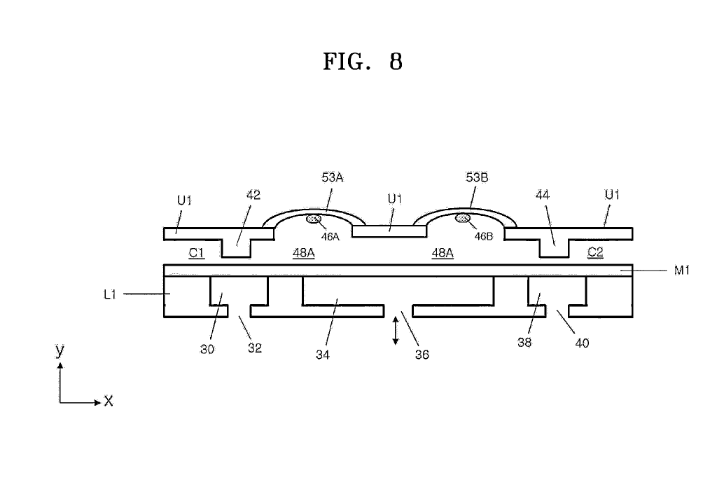

FIG. 8 is a cross-sectional view taken along the line 8-8′ of FIG. 7. Referring to FIG. 8, the third and fourth covers 53A and 53B are located on the dissolution chamber 48A. The third and fourth covers 53A and 53B may be considered convex in the Y-axis direction. The external sides of the third and fourth covers 53A and 53B may be considered convex in the Y-axis direction. The internal sides of the third and fourth covers 53A and 53B, which contact a solution that flows into the dissolution chamber 48A, may be considered concave in the Y-axis direction. A first solid reagent 46A may be located underneath the internal side of the third cover 53A. A second solid reagent 46B may be located underneath the internal side of the fourth cover 53B. The first and second solid reagents 46A and 46B may be the same or different reagents.

In the case where the first and second solid reagents 46A and 46B are disposed in the dissolution chamber 48A, a dissolving solution that flows into the dissolution chamber 48A may include both a target material for dissolving the first solid reagent 46A and a target material for dissolving the second solid reagent 46B. The dissolving solution may include only one target material that is capable of dissolving the first and second solid reagents 46A and 46B simultaneously.

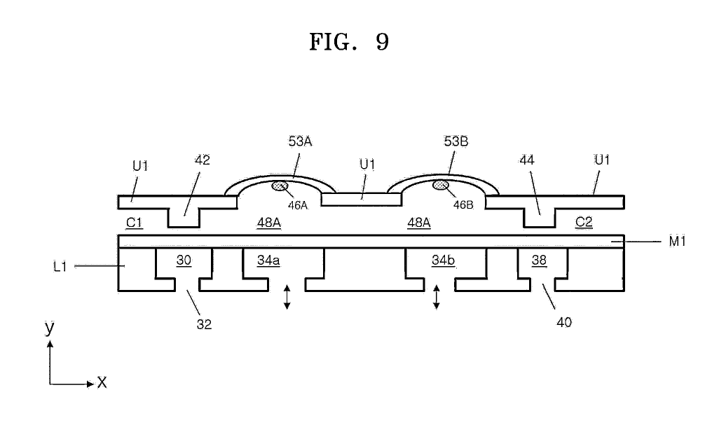

In FIG. 8, a plurality of pneumatic chambers may be formed instead of the second chamber 34 that is a pneumatic chamber. FIG. 9 illustrates a case in which a plurality of pneumatic chambers are formed instead of the second chamber 34 of FIG. 8.

Referring to FIG. 9, fourth and fifth chambers 34a and 34b are formed between the first and third chambers 30 and 38 and apart from each other. The fourth and fifth chambers 34a and 34b are located under the dissolution chamber 48A. The fourth chamber 34a corresponds to the third cover 53A, and the fifth chamber 34b corresponds to the fourth cover 53B.

In FIGS. 8 and 9, the third and fourth covers 53A and 53B may be replaced with covers having other forms. For example, the third and fourth covers 53A and 53B may each be replaced with the cover 51 of FIG. 6.

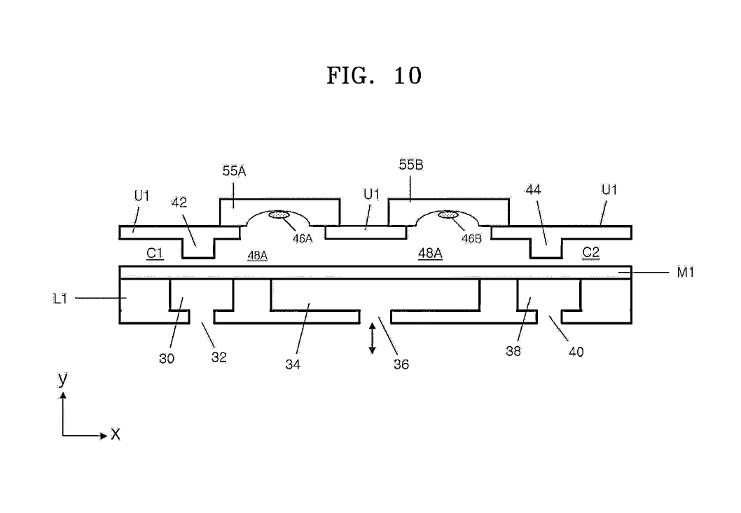

FIG. 10 illustrates a case in which the third and fourth covers 53A and 53B of FIG. 8 are replaced with fifth and sixth covers 55A and 55B, respectively. The shape of each of the fifth and sixth covers 55A and 55B may be the same as that of the second cover 51 of FIG. 6. The first solid reagent 46A is disposed underneath the internal side of the fifth cover 55A. The second solid reagent 46B is disposed underneath the internal side of the sixth cover 55B.

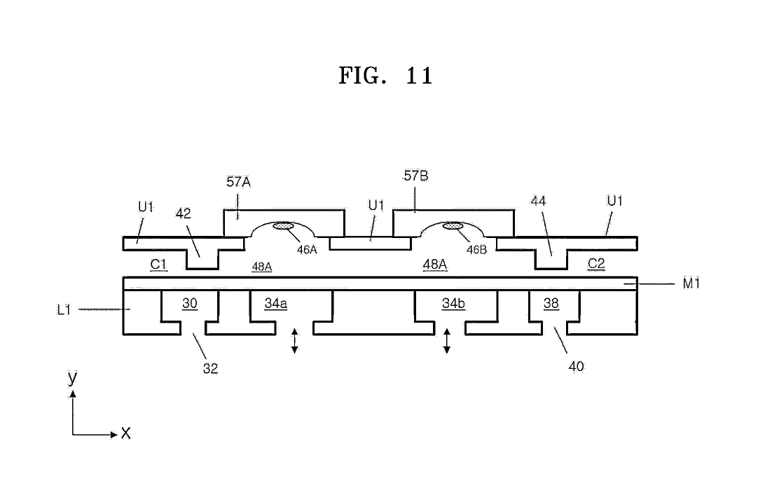

FIG. 11 illustrates a case in which the third and fourth covers 53A and 53B of FIG. 9 are replaced with seventh and eighth covers 57A and 57B. The shape of each of the seventh and eighth covers 57A and 57B may be the same as that of the second cover 51 of FIG. 6. The seventh cover 57A corresponds to the fourth chamber 34a, and the eighth cover 57B corresponds to the fifth chamber 34b. The first solid reagent 46A is disposed underneath the internal side of the seventh cover 57A. The second solid reagent 46B is disposed underneath the internal side of the eighth cover 57B.

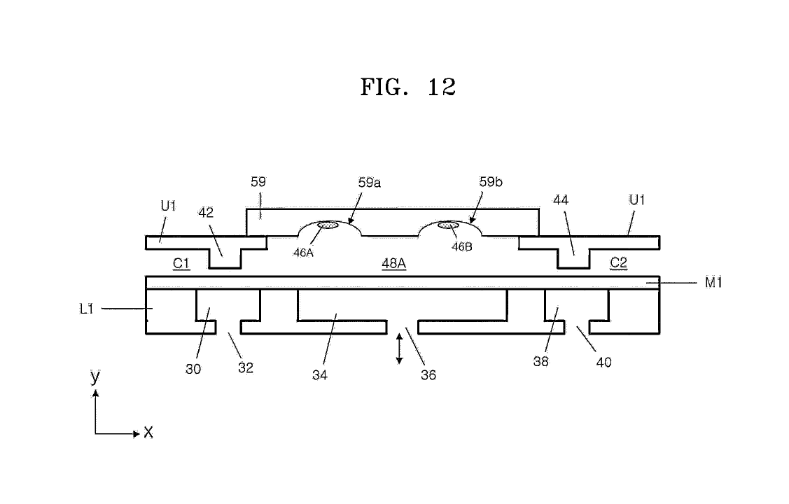

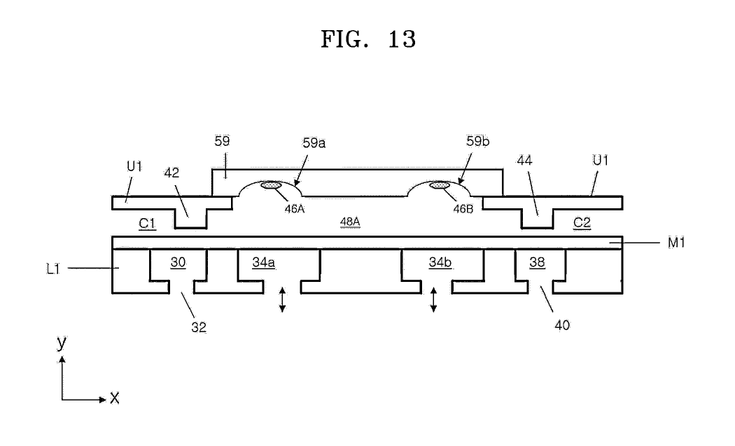

FIGS. 12 and 13 illustrate cases in which a plurality of curved surface portions are formed in a single cover.

Referring to FIG. 12, a single cover, i.e., a ninth cover 59, is disposed where a portion of the upper plate U1 is removed. The external side of the ninth cover 59 includes an upper side and lateral sides. The upper side of the ninth cover 59 is parallel with the upper side of the upper plate U1. The lateral sides of the ninth cover 59 may be perpendicular to the upper side thereof. The internal side of the ninth cover 59, which contacts a fluid that flows into the dissolution chamber 48A, includes first and second curved surface portions 59a and 59b. The first and second curved surface portions 59a and 59b are spaced apart from each other. The shapes of the first and second curved surface portions 59a and 59b may be the same as each other, but may be different from each other. The first and second curved surface portions 59a and 59b may be, for example, a concave side in the Y-axis direction. The first and second solid reagents 46A and 46B may be located underneath the first and second curved surface portions 59a and 59b, respectively. The ninth cover 59 may be disposed at a location that corresponds to the second chamber 34, i.e., a pneumatic chamber, included in the lower plate L1. The first and second curved surface portions 59a and 59b of the internal side of the ninth cover 59 may be located over the second chamber 34.

In FIG. 12, the second chamber 34 may be replaced with a plurality of pneumatic chambers, and FIG. 13 illustrates a case in which the second chamber 34 of FIG. 12 is replaced with two pneumatic chambers.

Referring to FIG. 13, the fourth and fifth chambers 34a and 34b are between the first chamber 30 of the lower plate L1 and the third chamber 38 of the lower plate L1. The fourth and fifth chambers 34a and 34b are apart from each other and apart from the first and third chambers 30 and 38. The fourth chamber 34a is disposed at a location that corresponds to the first curved surface portion 59a of the internal side of the ninth cover 59. The fifth chamber 34b is disposed at a location that corresponds to the second curved surface portion 59b of the internal side of the ninth cover 59.

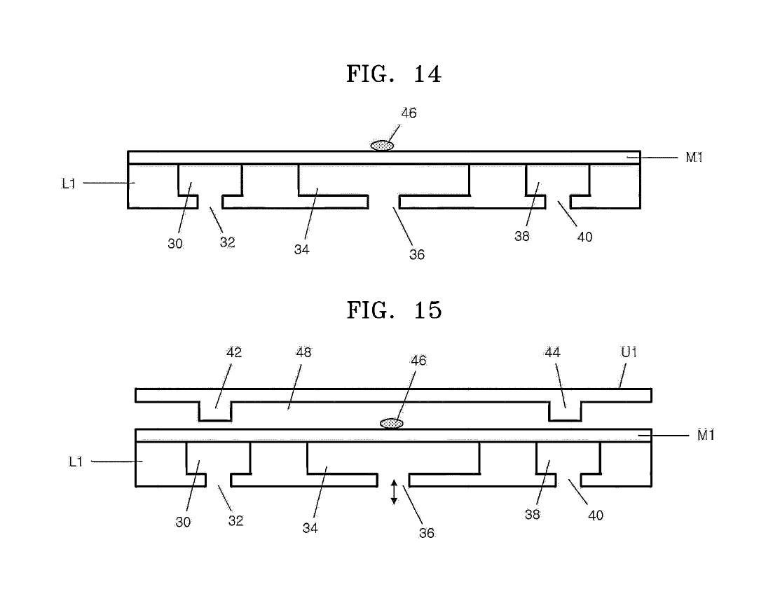

Next, a method of dissolving a solid reagent, according to an embodiment of the present invention, is described with reference to FIGS. 14 through 18. The method can be performed using, for instance, the solid reagent dissolving device described herein.

Referring to FIG. 14, a solid reagent 46 is disposed on an intermediate layer M1 after removing the upper plate U1 in the dissolving device of FIG. 1. The solid reagent 46 may be located on a portion of the intermediate layer M1, which covers a second chamber 34 of a lower plate L1. At this time, the solid reagent 46 may be located in a place that is opposite to an air inlet 36 of the second chamber 34. The solid reagent 46 may be formed by lyophilizing a liquid reagent after placing the liquid reagent in a predetermined location of the intermediate layer M1. The lyophilization may be performed by using a known method or apparatus.

The solid reagent 46 may include various components depending on a target material to be analyzed. For example, the target material may include target DNA, target RNA, a protein, or cell debris. If the target material is target DNA, the solid reagent 46 may include polymerase, a primer/probe, a buffer, and the like as components. In addition, the solid reagent may be a lyophilized PCR reagent. The PCR reagent may include polymerase, a primer/probe, dNTP, and a buffer. In addition, the solid reagent may be a lyophilized nucleic acid hybridization reagent, a ligation reaction reagent, a restriction enzyme reaction reagent, an in vitro transcription reaction reagent, or an in vitro translation reaction reagent.

Next, as illustrated in FIG. 15, the upper plate U1 is placed on the intermediate layer M1. At this time, the upper plate U1 is aligned so that a first protrusion 42 and a second protrusion 44 of the upper plate U1 correspond to a first chamber 30 and a third chamber 38 of the lower plate L1, respectively. If the upper plate U1 is aligned, the whole structure of the dissolving device becomes a three-layer structure as in FIG. 1, and the solid reagent 46 is located in the dissolution chamber 48 between the upper plate U1 and the intermediate layer M1.

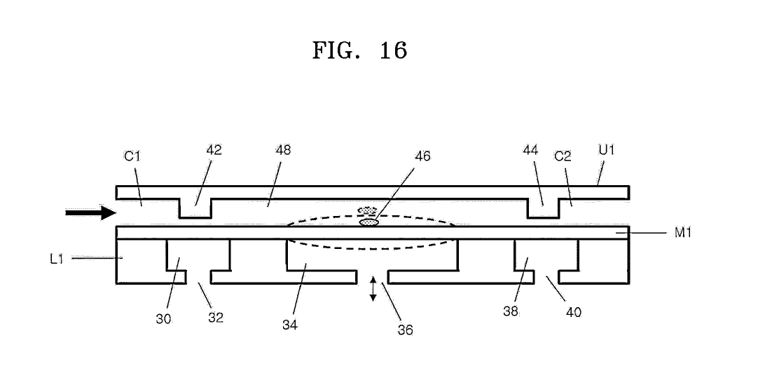

Next, referring to FIG. 16, after properly aligning the upper plate U1, a solution for dissolving the solid reagent 46 is injected to the dissolution chamber 48 through a first minute channel C1. The dissolving solution may have a characteristic that dissolves the solid reagent. The dissolving solution may be water, a solution of salt, and/or a buffer. The buffer may be properly selected depending on a selected reagent. The buffer may be a phosphate buffer solution (PBS) or a tris(hydroxymethyl)aminomethane (Tris) buffer. If the dissolving solution is filled in the dissolution chamber 48, the intermediate layer M1 is periodically or aperiodically vibrated. This vibration may be applied until the solid reagent 46 is dissolved. When the vibration is periodic, the number of vibrations, i.e., the vibration frequency, may be from about 0.001 Hz to about 100 kHz. The vibration may be generated by repeatedly pressuring and depressurizing the second chamber 34, i.e., a pneumatic chamber, of the lower plate L1. A pressurization of the second chamber 34 may be performed by supplying air pressure to the second chamber 34 by using an air pump that is connected to the lower opening 36 of the second chamber 34, which is an inlet. A depressurization of the second chamber 34 may be performed by using a depressurization pump. In another embodiment, the pressurization and the depressurization of the second chamber 34 may be performed by using an air pump.

A dashed line of FIG. 16 indicates a vibration of the intermediate layer M1 covering the second chamber 34. Depending on the vibration of the intermediate layer M1, the solid reagent 46 placed on the intermediate layer M1 and the dissolving solution supplied to the dissolution chamber 48 also are vibrated. During this vibration, the solid reagent 46 may be completely dissolved by rubbing against the dissolving solution.

Beads may be introduced into the dissolution chamber 48 prior to, or after, supplying the dissolving solution. In some embodiments, the beads do not chemically react with the solid reagent 46. The beads and the dissolving solution may vibrate inside the dissolution chamber 48 by vibration of the intermediate layer M1. The size of the beads may be larger than gaps between first and second protrusions 42 and 44 and the intermediate layer M1. As the beads are included in the dissolving solution, the solid reagent 46 may collide with the beads and rub against the dissolving solution during the vibration. Thus, a dissolving time of the solid reagent 46 may decrease in the presence of the beads and the dissolution of the solid reagent 46 may be more effectively performed to improve reproducibility, compared to when only the dissolving solution is used to dissolve the solid reagent 46 in the second chamber 48. The beads may be microbeads that are capable of being included in the dissolution chamber 48. The microbeads may have a diameter in the range of about 10 nm to about 1000 um, for example, about 1 μm˜100 μm. In addition, the lyophilization may be performed in a state in which the liquid reagent has been introduced into the dissolution chamber 48.

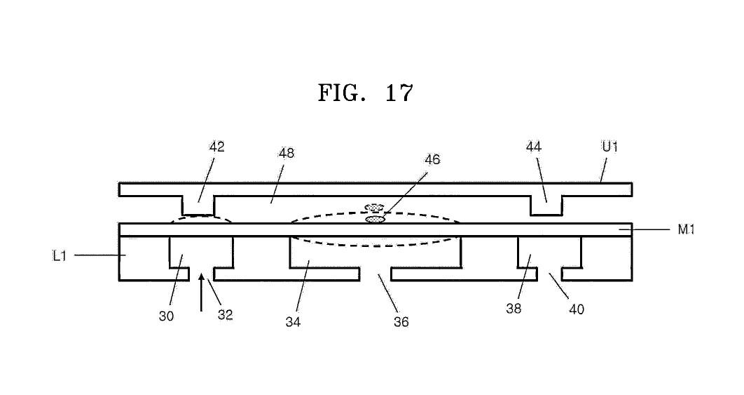

After supplying the dissolving solution in the dissolution chamber 48, the gap between the first protrusion 42 and the intermediate layer M1 may be closed and then the intermediate layer M1 may be vibrated, as shown in FIG. 17.

Referring to FIG. 17, after the dissolving solution is filled in the dissolution chamber 48, an air having a pressure higher than atmosphere pressure is supplied to the first chamber 30. Thus, a vibration plate, i.e., the intermediate plate M1, covering the first chamber 30 is pressured upwards and thus becomes convex, and contacts the first protrusion 42 of the upper plate U1. An air pump (not shown) may be connected to a lower opening 32 of the first chamber 30, which is an inlet. The air having a pressure higher than the atmosphere pressure may be supplied to the first chamber 30 by using the air pump. A dashed line convexly drawn between the first protrusion 42 and the intermediate layer M1 indicates that the intermediate layer M1 underneath the first protrusion 42 becomes convex upwards. As the intermediate layer M1 contacts the first protrusion 42, the gap between the first protrusion 42 and the intermediate layer M1 disappears, and the first minute channel C1 is closed. In this state, as explained with reference to FIG. 16, a dissolution operation of the solid reagent 46 may be performed by vibrating the intermediate layer M1 over the second chamber 34.

In FIG. 17, instead of closing the gap between the first protrusion 42 and the intermediate layer M1, the gap between the second protrusion 44 and the intermediate layer M1 may be closed to perform a dissolution process of the solid reagent 46.

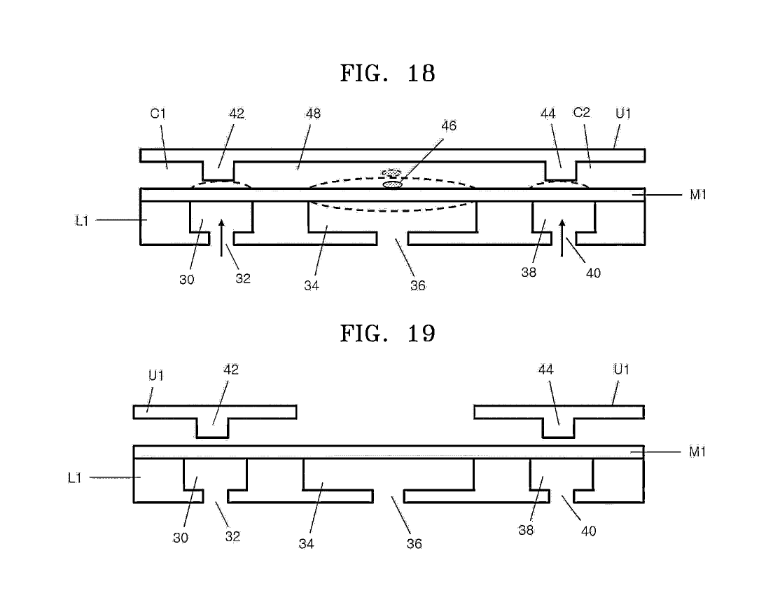

In addition, the dissolution process of the solid reagent 46 may be performed after closing all the gaps between the first and second protrusions 42 and 44 and the intermediate layer M1, as shown in FIG. 18.

Referring to FIG. 18, pressure (e.g., air pressure) higher than the atmosphere pressure may be supplied to the first and third chambers 30 and 38 after supplying the dissolving solution to the dissolution chamber 48. The pressure may be supplied by using an air pump connected to each of the first and second chambers 30 and 38, however the embodiments described herein are not limited to an air pump. Any known mechanism for supplying pressure may be used. As a result, the intermediate layer M1 over the first and third chambers 30 and 38 becomes convex upwards, as illustrated by a dashed line of FIG. 18, and thus contacts the first and second protrusions 42 and 44. Thus, the first and second minute channels C1 and C2 are closed. In this state, the dissolution process of the solid reagent 46 may be performed as previously described.

Also in a case where the second chamber 34 is replaced with a plurality of chambers, for example, the fourth and fifth chambers 34a and 34b of FIG. 4, the above-described method for dissolving the solid reagent 46 may be applied. In particular, the method of vibrating the intermediate layer M1 by using the second chamber 34 may be applied to each of the fourth and fifth chambers 34a and 34b.

Next, a method of dissolving a solid reagent, according to another embodiment of the present invention, is described with reference to FIGS. 19 through 21.

Referring to FIG. 19, an upper plate U1 from which a portion has been removed is aligned on the intermediate layer M1. The removed portion is a portion that may be detachably attached to the upper plate U1, and may be a portion of a dissolution chamber.

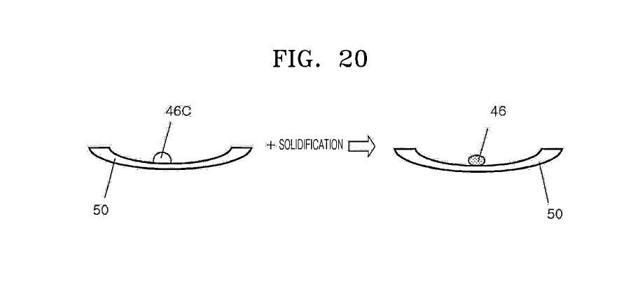

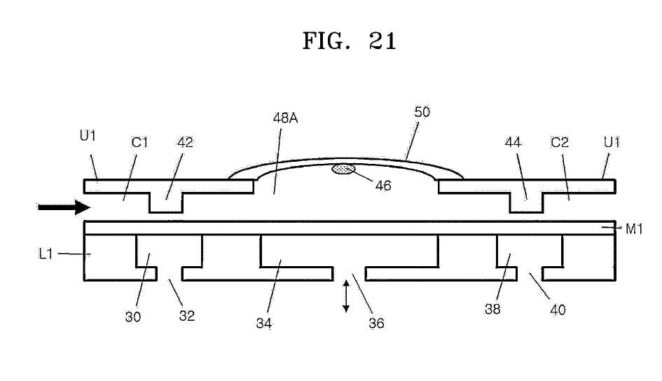

Referring to FIG. 20, a first cover 50—used as a cover at the location corresponding to that of the removed portion of the upper plate U1—is reversed, inverted, “flipped over”, turned “up-side down,” etc. In some embodiments, the second cover 51 of FIG. 6 may be used instead of the first cover 50. A prepared liquid reagent 46C is put on the center of the upper side of the reversed first cover 50. In this state, the liquid reagent 46C may be solidified, for example, by using a lyophilizing method. By the solidification, the liquid reagent 46C becomes a solid reagent 46. Next, the first cover 50 is reversed again, and positioned at the location corresponding to that of the removed portion of the upper plate, as illustrated in FIG. 21. The first cover 50 and the upper plate U1 may be coupled by using a coupling element, for example, a mechanical coupling element or an adhesive.

In this manner, a dissolution chamber 48A is formed under the first cover 50. After positioning the first cover 50 at a location corresponding to the removed portion of the upper plate U1, a solution for dissolving the solid reagent 46 is supplied to the dissolution chamber 48A through a first minute channel C1. Next, processes for dissolving the solid reagent 46 may be the same as those described with reference to FIGS. 16 through 18.

A cover, which has a plurality of curved surface portions in the internal side thereof, such as the third and fourth covers 53A and 53B of FIG. 8, the fifth and sixth covers 55A and 55B of FIG. 10, or the ninth cover 59 of FIG. 12, may be used instead of the first cover 50. In this case, after introducing different liquid reagents in the plurality of curved surface portions, different solid reagents may be formed in the plurality of curved surface portions by solidifying the different liquid reagents as described above.

In the case where the different solid reagents are formed in the different curved surface portions, a dissolving solution that is supplied to the dissolution chamber 48A may include respective target materials for dissolving the respective different solid reagents. The dissolving solution may include only one target material that is capable of dissolving the different solid reagents simultaneously.

In addition, in the method of FIGS. 19 through 21, a second chamber 34 corresponding to the dissolution chamber 48A may be replaced with a plurality of pneumatic chambers performing the same function as the second chamber 34, for example, the fourth and fifth chambers 34a and 34b of FIG. 4.

The foregoing embodiments have been described in reference to the use of a fluid, such as air, to pressurize or depressurize the pneumatic chamber. However, any fluid that can be flowed into and out of the chamber to cause the intermediate layer to deflect into or away from the second chamber can be used. Non-limiting examples of such fluids include air, as previously mentioned, as well as other gases, particularly gases that are inert with respect to the materials of the second chamber and the intermediate layer (or other components with which the gas may come into contact). Specific examples of such gases include, for example, argon or nitrogen.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A solid reagent dissolving device comprising:
a lower plate;
a flexible layer disposed on the lower plate; and
an upper plate disposed on the flexible layer, wherein the upper plate comprises
a plurality of channels;
a dissolution chamber in fluid communication with the plurality of channels; and
at least one protrusion that limits flow of fluid through at least one of the plurality of channels;
wherein the lower plate comprises a plurality of penetration holes in regions of the lower plate that correspond to the protrusion and the dissolution chamber of the upper plate, respectively; and
wherein the flexible layer covers each of the plurality of penetration holes, the plurality of channels, and the dissolution chamber.

2. The solid reagent dissolving device of claim 1, further comprising a cover positioned on the upper plate and covering at least a portion of the dissolution chamber.

3. The solid reagent dissolving device of claim 1, wherein a portion of the upper plate corresponding to the dissolution chamber is parallel with the flexible layer.

4. The solid reagent dissolving device of claim 1, wherein each penetration hole comprises an opening in an upper side of the lower plate and an opening on a lower side of the lower plate, wherein the diameters of the openings are equal to or different from each other.

5. The solid reagent dissolving device of claim 1, wherein the penetration hole in a region of the lower plate corresponding to the protrusion is a valve chamber for opening and closing a path between the protrusion and the flexible layer.

6. The solid reagent dissolving device of claim 2, wherein the at least one penetration hole in a region of the lower plate corresponding to the dissolution chamber is a pneumatic chamber that generates vibration of a portion of the flexible layer when pneumatic pressure is repeatedly applied.

7. The solid reagent dissolving device of claim 1, wherein a surface of the flexible layer facing the upper plate, surfaces of the plurality of channels, and internal surfaces of the dissolution chamber are hydrophobic.

8. The solid reagent dissolving device of claim 1, wherein a thickness of the flexible layer is from about 1 μm to about 1000 μm.

9. The solid reagent dissolving device of claim 6, wherein the cover is separable from the upper plate, and the internal side of the cover comprises at least one curved surface portion configured to accept a solid reagent.

10. The solid reagent dissolving device of claim 3, wherein the at least one penetration hole corresponding to the dissolution chamber comprises a pneumatic chamber that generates vibration of a portion of the flexible layer which corresponds to the dissolution chamber.

11. The solid reagent dissolving device of claim 9, wherein the cover comprises first and second covers that are apart from each other, and internal sides of the first and second covers comprise respective curved surface portions configured to accept a solid reagent.

12. A method of dissolving a solid reagent, the method comprising:
disposing the solid reagent in a dissolution chamber of the device of claim 1;
supplying a solution for dissolving the solid reagent to the dissolution chamber; and
vibrating the solution and dissolving the solid reagent.

13. The method of claim 12, wherein the solid reagent is a lyophilized reagent.

14. The method of claim 12, wherein disposing of the solid reagent in the dissolution chamber comprises:
disposing a liquid reagent in the dissolution chamber; and
lyophilizing the liquid reagent.

15. The method of claim 12, wherein disposing of the solid reagent in the dissolution chamber comprises:
separating a cover attached to the dissolution chamber from the dissolution chamber;
placing a liquid reagent on the cover separated from the dissolution chamber;
lyophilizing the liquid reagent; and
replacing the cover over the dissolution chamber, whereupon the lyophilized reagent is placed in the dissolution chamber.

16. The method of claim 12, wherein vibrating the solution for dissolving is facilitated by vibrating a portion of the flexible layer covering the dissolution chamber.

17. The method of claim 16, wherein the flexible layer is vibrated with a frequency in the range of about 0.001 Hz to about 100 k Hz.

18. The method of claim 16, wherein vibrating the flexible layer comprises repeating a process of raising or lowering a pressure in the penetration hole in the region of the lower plate corresponding to the dissolution chamber.

19. The method of claim 12, wherein vibrating the solution for dissolving comprises vibrating the solid reagent and the solution for dissolving.

20. The method of claim 12, further comprising:
before vibrating the solution for dissolving, blocking at least one portion of a channel connected to the dissolution chamber.

21. The method of claim 20, wherein blocking the at least one portion of the channel comprises applying pressure to a penetration hole in a portion of the lower plate corresponding to a protrusion.

22. The method of claim 12, wherein the solution comprises a target material that reacts with the solid reagent.

23. The method of claim 22, wherein the target material comprises target DNA, target RNA, protein, or cell debris.

24. The method of claim 12, wherein the dissolution chamber comprises beads that vibrate with the solution and aid in dissolving the solid reagent.

25. The method of claim 12, wherein a portion of the dissolution chamber is defined by a cover, the cover is separable from the dissolution chamber, and an internal side of the cover comprises at least one curved surface portion configured to accept a liquid reagent.

26. The method of claim 25, wherein the cover comprises first and second covers spaced apart from each other, and internal sides of the first and second covers comprise respective curved surface portions configured to accept different liquid reagents.

27. A method of dissolving a solid reagent, the method comprising:
disposing the solid reagent in a dissolution chamber of the device of claim 12;
supplying a solution for dissolving the solid reagent to the dissolution chamber; and
repeatedly applying pressure to the flexible membrane, thereby vibrating the solution and dissolving the solid reagent.
